(19) United States
(12) Patent Application Publication
(10) Pub. No.: US 2014/0106380 A1
(43) Pub. Date: Apr. 17, 2014

(54) METHODS FOR IDENTIFYING INHIBITORS OF ABETA42 OLIGOMERS

(71) Applicant: Merck Sharp & Dohme Crop., Rahway, NJ (US)

(72) Inventors: Alexander McCampbell, Chalfont, PA (US); William J. Ray, Lansdale, PA (US); Dawn M. Toolan, Gilbertsville, PA (US); Wei-Qin Zhao, North Wales, PA (US)

(73) Assignee: Merck Sharp & Dohme Crop., Rahway, NJ (US)

(21) Appl. No.: 14/106,468

(22) Filed: Dec. 13, 2013

Related U.S. Application Data
(63) Continuation of application No. 13/880,120.
(60) Provisional application No. 61/394,854, filed on Oct. 20, 2010.

(51) Int. Cl. G01N 33/566

(52) U.S. Cl. CPC G01N 33/566
USPC 435/7.92; 436/501

(57) ABSTRACT

The invention herein is directed to immunoassays for the detection of Aβ42 oligomers. The inventive assays are based on the observations herein that the presence of Aβ42 oligomers in a preparation is directly related to a decrease in a C-terminal (CT) immunosignal and a correlated increase in an N-terminal (NT) immunosignal, relative to the immunosignal generated in the absence of Aβ42 oligomers, in an Aβ42 CT and NT ELISA assay and an Aβ42 CT AlphaLISA assay. The invention herein involves the use of these assays alone or in combination to screen for inhibitors of Aβ42 oligomerization.

### CROSS-REFERENCE TO RELATED APPLICATIONS

This application is a Continuation of U.S. patent application Ser. No. 13/880,120, filed Apr. 18, 2013, which is a 371 of PCT Patent Application No. PCT/US2011/056349, filed Oct. 14, 2011, which claims benefit of U.S. Provisional Patent Application No. 61/394,854, filed Oct. 20, 2010, each of which is hereby incorporated by reference in their entirety herein.

### FIELD OF THE INVENTION

The present invention relates to immunoassays for identifying inhibitors of soluble oligomers of Alzheimer's disease related proteins.

### BACKGROUND OF THE INVENTION

Amyloid beta (Aβ) protein misfolding represents a primary molecular pathology in the brain of Alzheimer's disease (AD), the leading cause of age-related dementia. Aβis derived from the amyloid precursor protein (APP) via sequential proteolytic cleavage at the β and γ secretase sites to generate peptides of 38 to 43 amino acids in length, among which Aβ40 and Aβ42 are the two most common forms (Gandy et al., 1994, Neurobiol. Aging 15:253-256; Marotta et al., 1992, J. Mol. Neurosci. 3:111-125; Selkoe et al., 1996, Ann. N. Y. Acad. Sci. 777:57-64). While Aβ40 is more abundant in the normal brain, Aβ42 is believed to be the predominant form contributing to AD pathogenesis, due largely to its high propensity to aggregate (Gandy et al., 1994; Selkoe et al., 1996).

Research advances in the past decade have suggested that oligomers of Aβ42, rather than fibrils or plaques, are the major culprit responsible for a series of pathological changes at the molecular and synaptic level, including damages to the brain synaptic network (Oddo et al., 2006, J. Biol. Chem. 281:15990-1604; Glabe, 2005, Subcell. Biochem. 38:167-177; Klein et al., 2001, Trends Neurosci. 24:219-224; Walsh et al., 2005, Biochem. Soc. Trans. 33:1087-1090; Shankar et al., 2007, Nat. Med. 14:837-842; Lacor et al., 2007, J. Neurosci. 27:796-807; Lafaye et al., 2009, Mol. Immunol. 46:695-704; Lacor et al., 2004, J. Neurosci 24:10191-10200), that result in functional deficits, such as, impairment of synaptic plasticity (Shankar et al., 2008, Nat. Med. 14:837-842; Townsend et al., 2006, Ann. Neurol. 60:668-676;Walsh et al., 2002, Nature 416:535-539) and learning and memory (Balducci et al., 2010, Proc. Natl. Acad. Sci. USA 107:2295-2300; Shankar et al., 2008; Selkoe, 2008, Behav. Brain Res. 192:106-113). Because progressive synaptic degeneration underlies memory loss in the early stage of AD, targeting Aβ42 oligomer formation is a potential approach to protect synaptic structures from the toxicity of Aβ42 oligomers. To this end, much effort has been devoted to identifying small molecules that can interrupt the oligomerization of soluble Aβ42. Compounds that inhibit the formation of Aβ oligomers have also been shown to protect synapses against Aβ oligomer toxicity and improve cognition and learning deficits in AD transgenic animal models (Hawkes et al., 2010, Eur. J. Neurosci. 31:203-213; Townsend et al., 2006; McLaurin et al., 2000, J. Biol. Chem. 275:18495-18502).

In the past investigators focused largely on identifying compounds that inhibit the formation of large, β-sheet-rich, insoluble fibrils of Aβ. Oligomerization, as referred to herein, is an early to intermediate stage of Aβ misfolding. As the disease progresses, Aβ oligomers ultimately become larger aggregates, seen as amyloid deposits (or plaques) in the brain. Previous Aβ fibrillization inhibitors have been identified via assays using thioflavin derivatives or through the use of congo red, that show high binding affinity to Aβ fibril and plaques (Durairajan et al., 2008, Neurochem. Int. 52:742-750; Bartolini et al., 2007, ChemBioChem 8:2152-2161;Yang et al., 2005, J. Bio. Chem. 280:5892-5901; Joubert et al., 2001, Proteins, 45:136-143; Baine et al., 2009, J. Pept. Sci. 15:499-503; Chen et al., 2009, Bioorg. Med. Chem. 17:5189-5197; Sanders et al., 2009, Peptides 30:849-854), as well as, the β-structure of other aggregated proteins. However, compounds screened with these assays might not effectively control disease progression because they predominantly bind to fibrils and plaques and have little effect on toxic oligomer species. Moreover, these plaque-binding compounds may have the ability to dissolve insoluble Aβ aggregates, which has the potential to release active small oligomer species.

A major challenge to the detection and quantification of Aβ42 oligomers is that, in solution, Aβ42 species are highly heterogeneous in size and shape with continuous conversion occurring between monomer and oligomer species (Urbanc et al., 2010, Proc. Natl. Acad. Sci. USA 101:17345-17350; Walsh et al., 2009, FEBS J. 276:1266-1281; Teplow, 2006, Methods Enzymol. 413:20-33; Teplow et al., 2006, Acc. Chem. Res. 39:635-645). This metastable and polydispersed property makes quantification of Aβ42 oligomerization extremely difficult (Teplow et al., 2006; Teplow, 2006). Although a wide variety of technologies have been devoted to study the structure of Aβ oligomers (Teplow et al., 2006; Wu et al., 2009, J. Mol. Biol. 387:492-501; Baumketner et al., 2006, Protein Sci. 15:420-428; Bernstein et al., 2005, J. Am. Chem. Soc. 127: 2075-2084), at present there is no robust method that measures Aβ42 oligomerzation with reliability and high sensitivity.

Using a photo-induced cross-linking of unmodified proteins (PICUP) methodology, Bitan and colleagues have shown that Aβ42 preferentially forms paranuclei units composed of pentamer/hexamer species that act as building blocks for self-association of larger assemblies, comprising mostly dodecamers (Bitan et al., 2001, J. Biol. Chem. 276:35176-35184; Bitan et al., 2003, Proc. Natl. Acad. Sci. USA 100:330-335; Bitan and Teplow, 2005, Methods Mol. Biol. 299:3-9). The C-terminus of Aβ42 has been shown to play a critical role in oligomerization of Aβ42, with Ile⁴¹ being essential for paranuclei formation, as compared to Ala⁴² which is required for rapid self-association into larger assemblies (Bitan et al., 2003). In modeling systems, such as computational (Urbane et al., 2004, Proc. Natl. Acad. Sci., USA 101:17345-17350; Baumketner and Shea, 2005, Biophys J. 89:1493-1503; Baumketner et al., 2006, Protein Sci. 15-420-428) and electro-spray ionization ion-mobility mass spectrometry, (Baumketner and Shea, 2005; Bernstein et al. 2009, Nat. Chem. 1:326-331), it has been proposed that the C-terminal hydrophobic tail of Aβ42 is located in the center of a pentamer/hexamer, whereas the hydrophilic N-terminus is exposed on the surface of the oligomer. This prediction is consistent with in vitro data from experimentally produced globular oligomers (Barghorn et al., 2005, J. Neurochem. 95:834-847).

As such, based on the above, there is a need for improved assays that can detect and measure the spontaneous oligomerization of Aβ42 oligomers and to screen for inhibitors that can disrupt this initial process.

### SUMMARY OF THE INVENTION

The invention herein is directed to immunoassays for the detection of Aβ42 oligomers that are formed from the spontaneous oligomerization of Aβ42 in aqueous solution. The inventive assays are based on the observations herein that the presence of Aβ42 oligomers in a preparation is directly related to an increase in a C-terminal (CT) immunosignal and a correlated decrease in an N-terminal (NT) immunosignal in an Aβ42 CT and NT ELISA assay and an Aβ42 CT AlphaLISA assay. As such, the invention herein involves the use of these assays alone or in combination to screen for inhibitors of Aβ42 oligomerization.

In one embodiment the inventive assay comprises an Aβ42 C-terminal (CT) oligomer assay that comprises an ELISA using a capture antibody that recognizes an epitope in the N-terminal region of Aβ42 and an alkaline phosphatase (AP) conjugated detection antibody that recognizes an epitope in the C-terminal regional of Aβ42, that are reacted in the presence of an AP chemiluminescent substrate to produce a CT immunosignal, wherein said CT immunosignal will decrease, relative to the CT immunosignal generated in the absence of Aβ42 oligomers, when Aβ42 oligomers are detected. In a sub-embodiment of this assay, the capture and detection antibodies are 6E10 and 12F4, respectively.

In another embodiment the inventive assay comprises an Aβ42 N-terminal (NT) oligomer assay that comprises an ELISA using a capture antibody that recognizes an epitope in the N-terminal region of Aβ42 and an alkaline phosphatase (AP) conjugated detection antibody that recognizes an epitope in the N-terminal regional of Aβ42, that are reacted in the presence of an AP chemiluminescent substrate to produce a NT immunosignal, wherein said NT immunosignal will increase, relative to the NT immunosignal generated in the absence of Aβ42 oligomers, when Aβ42 oligomers are detected. In a sub-embodiment of this assay, the capture and detection antibody are 6E10.

In still another embodiment the inventive assay comprises an Aβ42 C-terminal (CT) oligomer assay that is a bead based proximity assay. This embodiment uses an AlphaLISA assay format comprising simultaneously incubating i) a streptavidin coated donor bead, that binds to a biotinylated Aβ antibody that recognizes an epitope both in Aβ42 and Aβ40, ii) an acceptor bead conjugated to a second antibody, that recognizes an epitope at the C-terminal region of Aβ42, and iii) one or more samples of Aβ42, to form a reaction mixture, and incubating said reaction mixture with a second streptavidin donor bead that binds to said biontinylated Aβ antibody, to produce a CT immunosignal, wherein said CT immunosignal will decrease, relative to the CT immunosignal generated in the absence of Aβ42 oligomers, when Aβ42 oligomers are detected. In a sub-embodiment of this assay, the donor beads are conjugated to streptavidin and the acceptor beads are conjugated to the anti-Aβ42 CT antibody.

In a further embodiment of the Aβ42 C-terminal AlphaLISA oligomer assay, the reaction mixture is analyzed in the presence of one or more test compounds, wherein a compound that results in a CT immunosignal that is increased more than three standard deviations from the CT immunosignal of a control is an Aβ42 oligomer inhibitor.

### BRIEF DESCRIPTION OF THE DRAWINGS

FIGS. 1A-1C are schematic illustrations of the Aβ42 immunoassays described herein. FIG. 1A is an illustration of an Aβ42 C-Terminal (CT) ELISA showing the loss of CT immunosignal following Aβ42 oligomerization. The assay uses 6E10, immobilized onto an ELISA plate, as the capture antibody and 12F4 as the detection antibody, which specifically recognizes the CT of Aβ42. Upon oligomerization of Aβ42, the CT is buried within the center of the oligomer and becomes inaccessible, resulting in a reduced CT immunosignal. FIG. 1B is an illustration of an Aβ42 CT AlphaLISA assay (Perkin Elmer, 2008) showing the loss of CT immunosignal following Aβ42 oligomerization. Only the monomeric form of Aβ42 can bind the anti-CT acceptor bead, which upon binding emits a signal following excitation of the Streptavidin donor bead (left image). Upon oligomerization, the anti-CT acceptor bead can no longer to bind Aβ42 (right image), resulting in the loss of emission, i.e. no CT signal. FIG. 1C is an illustration of an N-Terminal (NT) ELISA showing the positive correlation of the immunosignal with Aβ42 oligomerization. The monomer Aβ42 molecules were captured by the antibody, 6E10, which was immobilized onto an ELISA plate (left image). A subsequent application of a second 6E10 antibody, labeled with alkaline phosphate (AP), AP-6E10, was unable to detect monomeric Aβ42, which is attributed to the occupancy of the same epitope by the capture 6E10 antibody (left image). AP-6E10 was able to detect oligomers of Aβ42 in that it can bind to the N-terminals of the Aβ42 oligomers exposed at the surface of (right image), which results in increased emissions, i.e. a high NT signal.

FIGS. 2A-2C are representations of the Aβ42 oligomers described herein. FIG. 2A represents Atomic Force Microscopy (ATM) images of Aβ42 monomers (panel 1) and Aβ42 oligomers (panels 2-5, increasing amplitude). The Aβ42 oligomers, prepared as described herein in Example 1, appear as particles with heterogeneous size and shapes (panels 3-5). FIG. 2B represents images obtained from a Western blot of Aβ42 monomers (M) and Aβ42 oligomers (O). The immunosignals were detected with a combination of biotin labeled 6E10 and 4G8 antibodies. The Aβ42 oligomer preparation (O) showed multiple higher order species ranging from 30 to >100 kDa detected on Western blot following SDS polyacrilamide gel electrophoresis (SDS-PAGE), whereas the control showed mainly monomer and low order Aβ42 species. A less exposed image (boxed) showed that the amount of the lower order species (monomer, trimer and tetramer) present was reduced in the oligomeric (O) samples. FIG. 2C shows the binding of Aβ42 oligomers to cultured hippocampal neurons. Oligomer binding shows a punctate pattern along the dendritic tree (arrows pointing to bound oligomers).

FIGS. 3A-3D are graphical representations of the immunosignal changes following oligomerization as measured in CT and NT ELISA assays. FIG. 3A represents an Aβ42 CT ELISA showing the effect of Aβ42 concentration on Aβ42 monomers (▪) and the formation of Aβ42 oligomers () with a concomitant decrease in the CT immunosignal. FIG. 3B represents an Aβ42 NT ELISA showing the inverse change upon oligomerization, with an increase in NT immunoreactivity in oligomerized Aβ42 () as compared to Aβ42 monomers (▪). FIG. 3C represents the inverse Aβ42 CT and NT immunoreactivity changes in a time course oligomerization reaction. FIG. 3D represents a sequential multiplex CT and NT ELISA showing an increase in the NT/CT immunosignal ratio for oligomers () as compared to monomers (▪).

FIGS. 4A-4B are graphical representations of the immunosignal changes following oligomerization as measured in an AlphaLISA assay. FIG. 4A represents higher AlphaLISA signals for monomers (▪) and reduced AlphaLISA signals for oligomers (). FIG. 4B represents the sensitivity of the AlphaLISA CT immunoassay. The results showed decrease in the CT immunosignal upon Aβ42 concentration dependent oligomer formation. When tested at varying concentration of Aβ42 in the assay (: 1 nM; ▪: 5 nM; ▴: 10 nM), the AlphaLISA CT assay was sensitive to detect oligomerization signals at as low as 1 nM Aβ42 .

FIGS. 5A-5B are graphical representations showing the changes in the CT immunosignal for Aβ42 oligomers in an AlphaLISA assay with the addition of inositol isomers. Scylloinositol (▪: SI) induced dose-dependent increases in the Aβ42 CT immunosignal in various buffers (FIG. 5A) (: PBS; ▪: NB; ▴: MEM), which was not observed with its stereoisomers myo-inositol (: MI) and chiro-inositol (▴: CI) (FIG. 5B).

FIGS. 6A-6B are graphical representations showing the changes in the CT and NT immunosignal for Aβ42 oligomers in an AlphaLISA assay with the addition of SI. SI induced an increase in CT immunosignal (FIG. 6A) and a corresponding decrease in NT immunosignal (FIG. 6B), suggesting that SI shifted the metastability of Aβ42 oligomers towards Aβ42 monomer.

FIGS. 7A-7B are graphical representations showing the changes in the CT and NT immunosignal for Aβ42 oligomers in an AlphaLISA assay with the addition of Aβ42 Fibrillogenesis Inhibitor Peptide IV (P-IV). P-IV induced a dose-dependent increase in the CT immunosignal (FIG. 7A). The increased CT immunosignal was correlated with a decrease in the NT immunosignal (FIG. 7B).

FIG. 8 is a graphic representation of a dynamic light scattering (DLS) plot of Aβ42 oligomerization. DLS directly measures the size of particles in solution, which provides a method for validating the presence of Aβ42 oligomers without further immunoreactions. When measured at 30 minutes following oligomerization, DLS detected an Aβ peak between 1-10 nm in radius (first peak). As the oligomerization time is prolonged, the peak shifted to greater sizes (10 nm and 100 nm in radius).

FIGS. 9A-9D are graphical representation of a high throughput screen (HTS) of Aβ42 inhibitors using an automated CT AlphaLISA assay. FIGS. 9A (Compound Class I) and 9C (Compound Class II) show representative plates in a three dose, 10 μm per dose, primary screen of a representative compound of Class I and Class II (Compound A and Compound B, respectively) and their corresponding dose response reaction plots (FIGS. 9B and 9D). In the primary screen a standard deviation greater than 3 times standard error (3× SD) was set as a cutoff. Compounds producing a CT signal with more than 3× SD would be selected as potential Aβ42 oligomer inhibitors. Once identified (FIG. 9A or 9C), the hits were confirmed with a dose-response assay (FIGS. 9B and 9D). Aβ42 oligomer inhibitors show dose-dependent efficacy in oligomerization inhibition.

FIG. 10 is a graphical representation of a CT AlphaLISA assay done with two capture/detection pairs, 4G8-6E10 and 4G8-12F4, showing that a known Aβ42 oligomer inhibitor (Compound C) does not affect the total amount of Aβ42 transferred from the oligomerization plate to the assay plate as shown by the 4G8-6E 10 pair of antibodies. Conversely, in the presence of Compound C, the transferred Aβ42 showed a markedly higher CT immunosignal (as shown by the 4G8-12F4 antibody pair), indicating inhibition of oligomerization by the compound.

### DETAILED DESCRIPTION OF THE INVENTION

Increasing evidence from both in vivo and in vitro studies suggests that accumulation of Aβ42 oligomers in the brain is a proximate contributor to the etiology of Alzheimer's disease (AD). Small molecule compounds that inhibit Aβ42 oligomerization reduce brain amyloid deposition in AD transgenic mouse and protect neurons from the action of Aβ oligomers (McLaurin et al., 2000, J. Biol. Chem. 275:18495-18502; Hawkes, et al., 2010. Eur. J. Neurosci. 31:203-213; Townsend et al., 2006, Ann. Neurol. 60: 668-676). Conversely, reduction of amyloid beta peptide 1-42 (A(342) deposits in the form of plaques without a concurrent decrease in Aβ42 oligomers was not effective in treating memory deficits in animal models (Head, E. et al., 2008, J. Neurosci. 28, 3555-3566). Thus, inhibition of Aβ42 oligomerization has been proposed as a therapeutic strategy for AD. However, due to the highly complex biochemical properties of the Aβ42 peptide, assays that measure early stage Aβ42 oligomerization for high throughput drug screening are currently unavailable.

Aβ42 is a self-associating amphipathic peptide with polar side chains located in its N-terminal (NT) region and non-polar side chains in its C-terminal (CT) region. Multiple in vitro and in silico studies have generated a consistent conformational model of Aβ42 oligomers in which the N-termini are exposed at the oligomer surface, whereas the C-termini are hidden in the center of the complex. The presence of extremely hydrophobic Ile⁴¹ and Ala⁴² at the C-terminus plays an important role in the oligomerization of Aβ42, which differs from Aβ40 by forming pentamers and hexamers (Bitain et al., 2003, Proc. Natl. Acad. Sci. U.S.A 100: 330-335), due to an intra-molecular turn at Gly³⁷-Gly³⁸, resulting in the hydrophobic C-terminal being situated in the center and the unstructured N-terminus at the periphery of the oligomer (Urbane et al., 2004, Proc. Natl. Acad. Sci. U.S.A. 101:17345-17350). It should be noted that the Aβ42 oligomers referred to herein are the result of a spontaneous, self-induced, aggregation process, such as those produced in an aqueous solution, such as PBS and neurobasal medium, according to the protocol of Example 1, and are distinct from previously reported non-fibrillar forms of Aβ42 oligomers, namely amyloid-derived diffusible ligands (ADDLs), the preparation of which requires the use of particular medium, such as Ham's F12 (Sigma-Aldrich Corp., St. Louis, Mo.), and treatment, for example, 5 mM in DMSO before oligomerization and high speed centrifugation, to isolate globular soluble oligomers. Subsequent studies, using biochemical (Barghorn et al., 2005, J. Neurochem. 95:834-847), biophysical (Ahmed et al., 2010, Nat. Struct. Mol. Biol. 17: 561-567), and ion mobility mass spectrum approaches have demonstrated a similar conformation for all forms of Aβ42 oligomers, including globular oligomers, pentamers, hexamers and dodecamers, in which the C-terminal tail is buried in the center and the N-terminus extends out from the surface of the oligomer.

Based on this conformational model, Applicants proposed that once oligomerized, the immunoreactivity of the Aβ42 oligomers could be detected and, when measured with specific antibody capture-detection ELISA formats, would show correlated, inverse changes at the N-termini and C-termini, with an increase in immunoreactivity at the N-terminus (NT) coupled with a simultaneous decrease in the immunoreactivity at the C-terminus (CT). Accordingly, Applicants herein have developed a highly sensitive immunoassay to detect and measure the early, spontaneous oligomerization of Aβ42 in vitro. Such an assay can be used in a high throughput screen format to identify compounds and peptides that can be used as Aβ42 oligomer inhibitors. Such inhibitors can be used as therapeutics for the prevention and treatment of diseases in which Aβ42 oligomers are associated, such as, but not limited to, Alzheimer's disease and other forms of dementia (e. g. mild cognitive impairment and Lewy body dementia), Down's syndrome, and Parkinson's disease.

As described herein, Applicants have confirmed the structural arrangement of Aβ42 oligomers using a CT and a NT Aβ42 assay. Based on this Aβ42 structural arrangement, novel assays have been developed to monitor oligomerization and de-oligomerization of Aβ42 using measurement of the loss or gain of the CT immunosignal. Under the experimental conditions used herein (Example 1), Aβ42 formed globular or annular oligomers with an average size of 13.6 nm. On Western blots, the relatively weaker and diffusible staining at higher molecular weights as compared to low order species suggests these are not insoluble aggregates, but early stage Aβ42 oligomers. The Aβ42 CT ELISA and AlphaLISA assays as described herein have been shown to be highly sensitive assays that can distinguish Aβ42 oligomers from monomers at low nM concentrations. The assays are also highly reliable in that a CT antibody can only bind to unfolded Aβ42 to generate an immunosignal. Conversely, the increase in the NT immunosignal provides strong verification of the presence of oligomers, rendering the assay relatively error proof, in that it excludes false negative or positive results due to a difference in the amount of Aβ42 present in the assay.

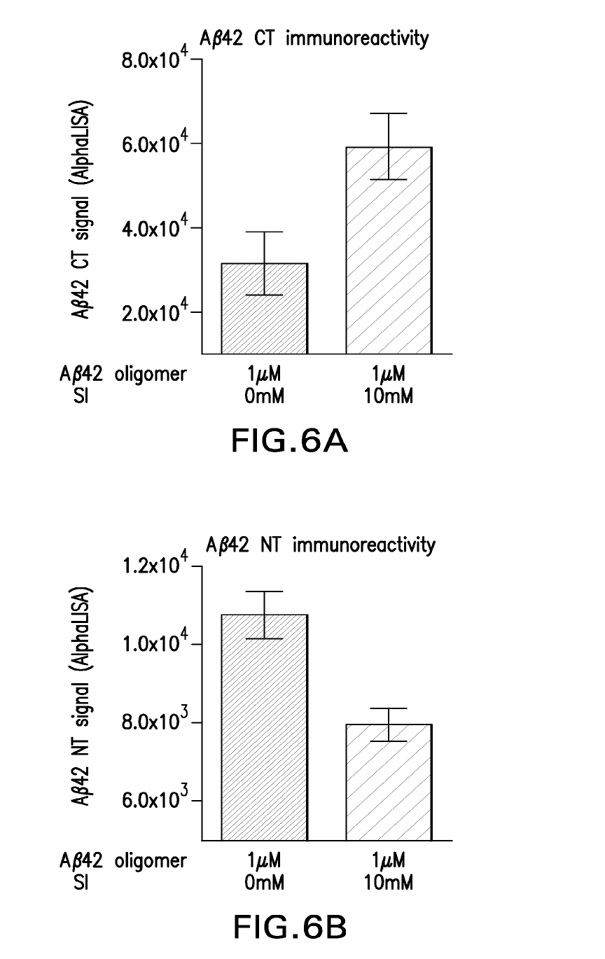

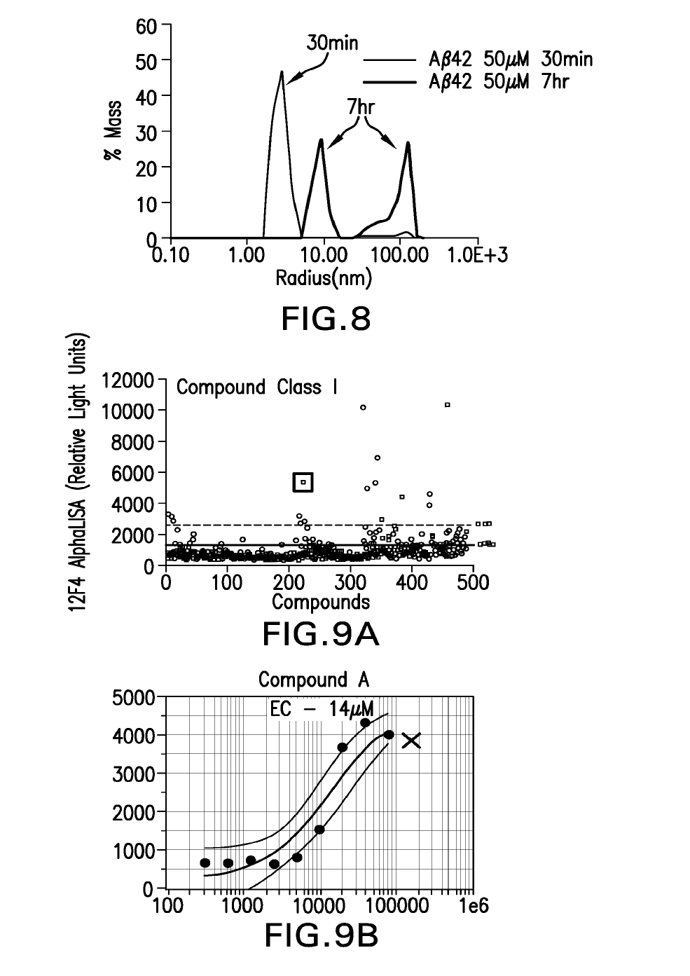

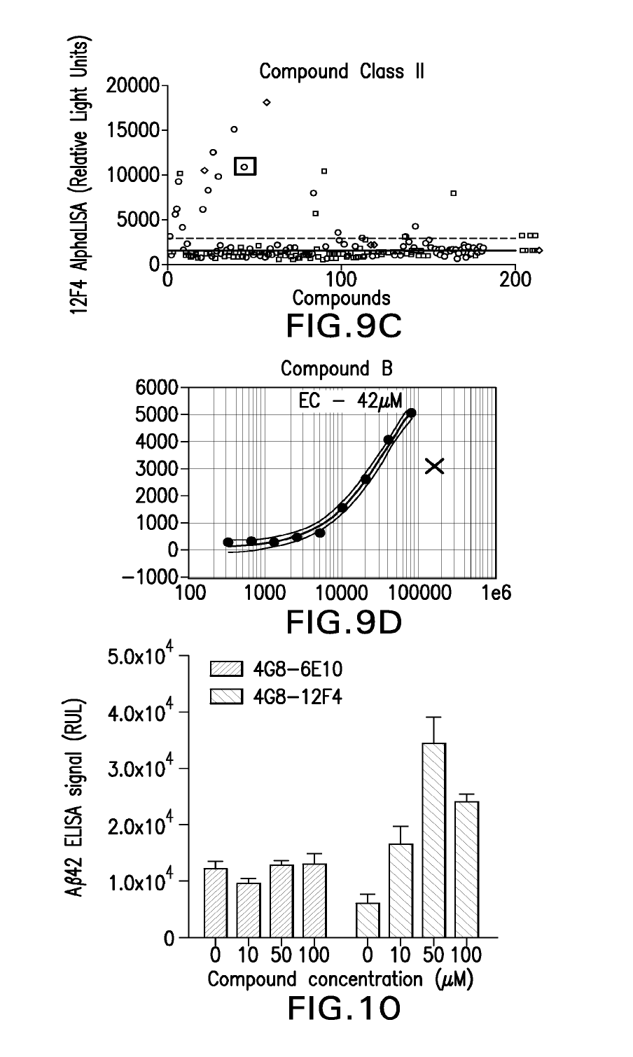

These properties are also indicative that the combination of the Aβ42 CT and NT assays is a robust tool to monitor in vitro oligomerization of Aβ42 and to identify small molecules and/or peptides that interfer with oligomerization. Known Aβ42 oligomer inhibitors, such as scyllo-inositol and the inhibitory Peptide IV (P-IV), produced a dose-dependent increase in the CT immunosignal and a corresponding decrease in the NT immunosignal. Scyllo-inositol not only inhibited oligomerization at the beginning of Aβ42 oligomerization, but was also shown to “de-oligomerize” oligomerized peptides (FIGS. 6A & 6B). Without wishing to be bound by any theory, Applicants herein showed that scyllo-inositol binds to monomers and/or stabilized lower order oligomers (McLaurin et al., J. Biol. Chem. 275:18495-18502; Townsend et al., Ann. Neurol. 60:668-676), which removed these species from the Aβ42 equilibrium, and then shifted the equilibrium toward monomer Inhibition of oligomerization by scyllo-inositol was also confirmed by DLS in which the size of the Aβ42 oligomers was reduced in the presence of scyllo-inositol (FIG. 8). The effectiveness of the inventive assay was further demonstrated in an automated high throughput screening (HTS) CT AlphaLISA assay (FIGS. 9A-9D) in which Applicants identified a small number of Aβ42 oligomer inhibitory small molecules by screening more than two thousand compounds from different structural classes. The specificity and validity of the hits have been verified with secondary immunoassays (FIG. 10). Based on their structural confirmation, it is more probable than not that the CT AlphaLISA assay can detect the formation of pentamers and higher order oligomer species, but it is not clear whether the assay can distinguish among lower order oligomer species, such as dimers, trimers and tetramers, and monomers. Notwithstanding, the HTS CT AlphaLISA assay was capable of detecting changes in oligomerization at 1 nM Aβ42, a concentration at which Aβ42 is unlikely to form larger insoluble aggregates. This finding suggests that the inhibitors, identified from the HTS assay, interfere or inhibit early oligomerization of Aβ42.

Accordingly, Applicants herein have developed a highly sensitive Aβ42 immunoassay to measure the early, spontaneous, oligomerization of Aβ42 in vitro. Using both sandwich ELISA and AlphaLISA assays, Applicants observed a reduction in the CT immunoreactivity for Aβ42 oligomers as compared to that for Aβ42 monomers. This reduction in CT immunoreactivity was accompanied by a concomitant increase in NT immunoreactivity. Applicants have also found using the assays described herein that scyllo-inositol, an isomer of inositol and a known small molecule Aβ42 oligomer inhibitor, showed a dose-dependent effect on unmasking the Aβ42 CT epitope. After verification with multiple methodologies the immunoassay was automated, which can be used as a highly reproducible and an effective method for high throughput screening (HTS) of small molecule compounds that inhibit Aβ42 oligomerization. Unlike thioflavin-T and congo red assays that had previously been used to detect Aβ42 oligomers, assays that rely on detecting insoluble amyloid plaques at micromolar concentrations, the inventive immunoassay, based on the inverse correlation between the CT and NT immunoreactive signals, can detect early stage oligomers formed from the spontaneous aggregation of Aβ42. The inventive immunoassay generates a robust signal that can be used to distinguish between Aβ42 monomers and Aβ42 oligomers present at concentrations as low as 1 nM. Moreover, the results from the inventive assay confirmed the structure of Aβ42 oligomers previously proposed by theoretical models. Thus, the invention herein offers a method for high throughput screening (HTS) for small molecule inhibitors of Aβ42 oligomerization.

The term “standard conditions” or “standard oligomerization conditions” refers to a process for the preparation of Aβ42 oligomers using synthetic human Aβ42 peptide, such as those of Example 1. Standard oligomerization conditions are as follows. A synthetic Aβ42 peptide is treated with hexafluoroisopropanol (HFIP) to breakdown any secondary structures. After HFIP is vaporized, Aβ42 is dissolved in dimethyl sulfoxide (DMSO) to make a 1 mM stock solution. The Aβ42 DMSO stock solution is used to make various concentrations (ranging from 100 μM to 1 nM) of Aβ42 solutions with aqueous solutions including, but not limited to, phosphate buffered saline (PBS), neurobasal medium (NB), and minimum essential medium (MEM). Oligomerization is performed at either room temperature or 37° C. for 30 to 180 minutes for the ELISA and AlphaLISA assays. To evaluate compounds or peptides as Aβ42 oligomer inhibitors, oligomerization is carried out in the presence of the test compounds under above conditions. The oligomerized samples are placed on ice for 1 to 2 hours to allow for a stable equilibrium before samples are subjected to the CT and NT assays.

The term “Aβ42” as used herein refers to the amyloid beta peptide comprising residues 1-42. This peptide is cleaved in a multi-step process from the amyloid precursor protein (APP) regardless of APP isoform.

The term “oligomer” or “Aβ42 oligomer” as used herein refers to the multiple species amyloid beta aggregate preparation formed from the early, spontaneous aggregation of Aβ42 in an aqueous solution, such as those produced from the method of Example 1. Such species include, but is not limited to, globular and proto-fibril species and mixtures thereof.

The term “pre-aggregated” or “pre-oligomerized” as used herein refers to formation of Aβ42 oligomers under standard conditions prior to addition of testing compounds. The term “non-aggregated” or “non-oligomerized” as used herein refers to monomer forms of Aβ42.

### Models for Assessment of Aβ42 Oligomers

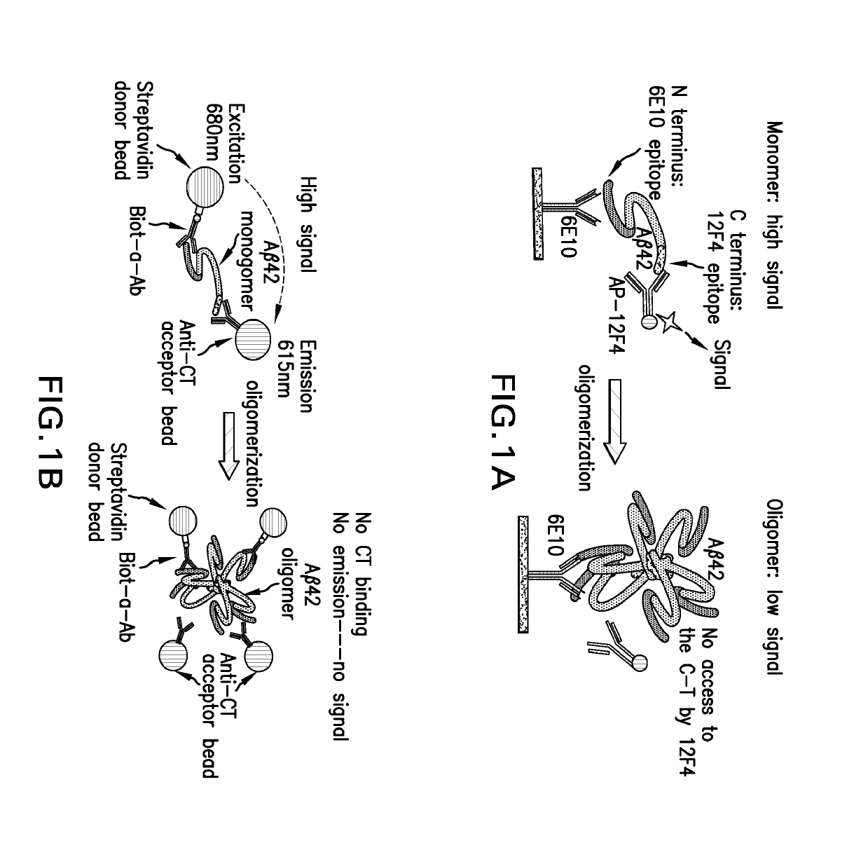

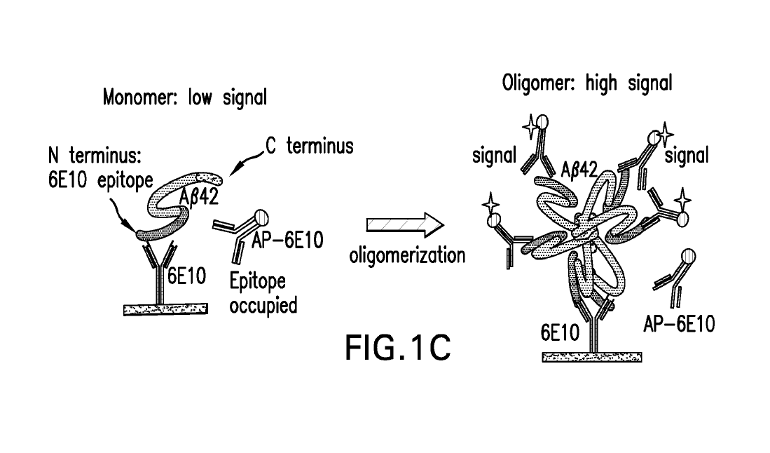

Based on the conformational model for oligomerized Aβ42, Applicants proposed that changes in immunoreactivity of the N- and C-termini could be used for assessing the oligomeric state of Aβ42 and for screening of Aβ42 oligomerization inhibitors. As illustrated in FIG. 1A, monomeric Aβ42 peptide (left image) (Aβ42) was detected in a sandwich ELISA with a capture antibody, 6E10, that binds to the N-terminus (NT), and a detection antibody, 12F4, that binds at the C-terminus (CT). In this instance, there was a direct correlation of the immunoreactivity, i.e. the CT immunosignal detected, of Aβ42 monomer with the amount of the monomer peptide present. When Aβ42 oligomerized (FIG. 1A, right image), immunoreactivity, i.e. the CT immunosignal, decreased as the CT of Aβ42 became cryptic or “hidden” within the oligomer center, such that it has limited or no availability for binding to the CT antibody, i.e. it is less accessible to the detection antibody.

The same principal was applicable to enable the use of an AlphaLISA (PerkinElmer, Waltham, Mass.) assay, which offered several advantages over a sandwich ELISA, including, higher sensitivity, low background, no wash step and a short assay time (Eglen et al., 2008, Curr. Chem. Genomics 1:2-10). As illustrated in FIG. 1B, similar results were observed in the Aβ42 CT immunosignal upon Aβ42 oligomerization in an AlphaLISA CT assay. Conversely, an assay format that measured NT immunoreactivity (Gandy et al., Ann. Neurol. 2010 68: 220-30) resulted in an increase in the CT immunosignal (FIG. 1C). In this latter format, an antibody recognizing an epitope in the NT (1-16 amino acid of Aβ42) was used as both the capture and the detection antibody, which resulted in little or no signal for Aβ42 monomers as the epitope is already occupied by the capture antibody (FIG. 1C, left image). In an NT assay, as the NT of oligomerized Aβ42 is exposed on the surface, the immunoreactivity increased as additional N-termini were made available for binding of the detection antibody (FIG. 1C, right image).

### Verification of Aβ42 Oligomer Preparations

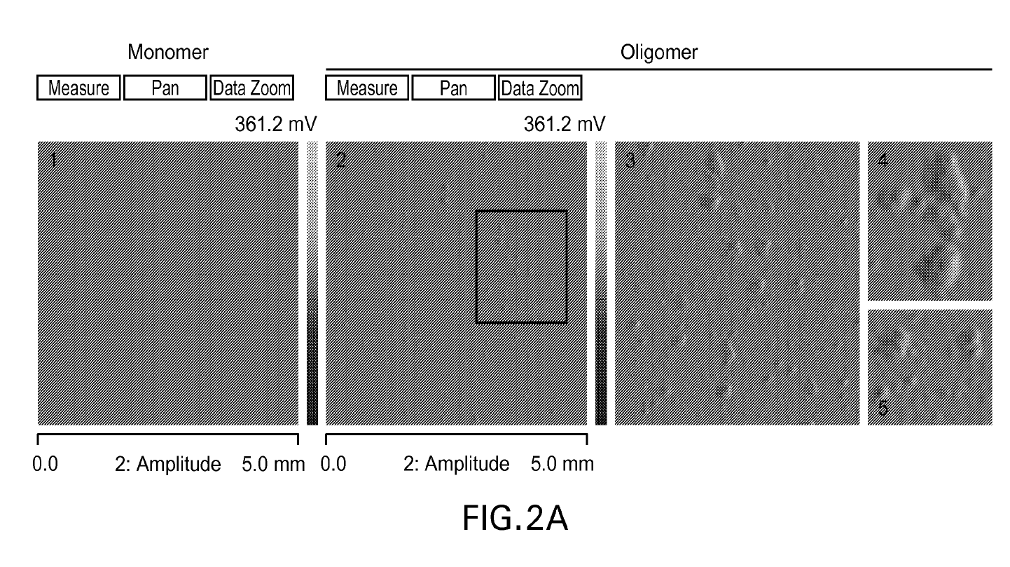

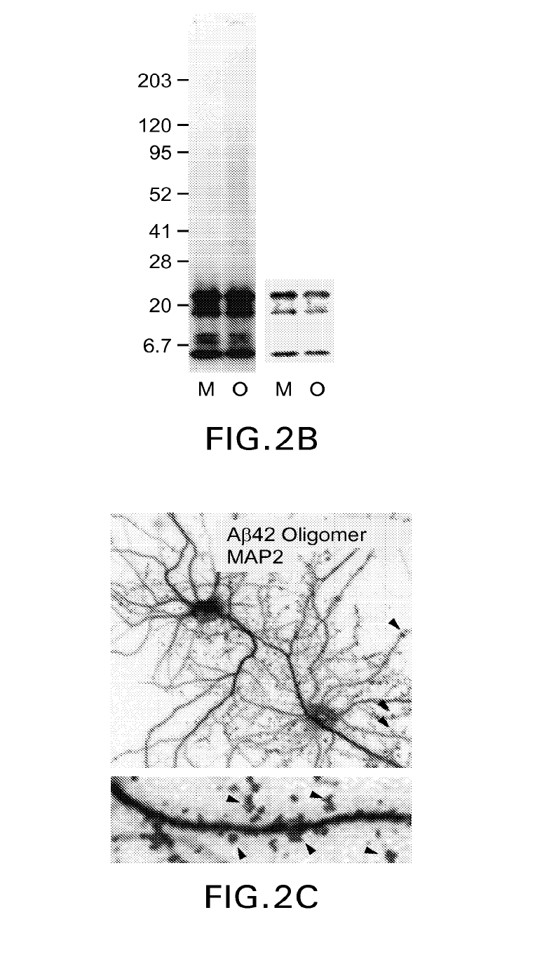

Aβ42 oligomer samples prepared under the standard oligomerization conditions described here were assayed for the presence of Aβ42 oligomers. When subjected to atomic force microscopy, the non-oligomerized Aβ42 showed very few visible particles on the mica sheet (FIG. 2A, monomer, panel 1). Oligomerized Aβ42 presented as numerous particles with heterogeneous shapes and sizes (FIG. 2B, oligomers, shown with increasing amplification from panels 2 to 5). While some were globular, many showed annular morphology distributed either individually (FIG. 2A, panel 3), or arranged in a short chain within a small cluster (FIG. 2A, panel 4). The globules had an average diameter of 13.6 nm (SD=3.6; n=194) and an average height of 2.8 nm (SD=1.5; n=194). The morphology of the spontaneous Aβ42 oligomers herein was different from the soluble, non-fibrilar, Aβ42 oligomer preparations of Chromy et al., 2003, Biochemistry 42:12749-12760 and Lambert et al., 1998, Proc. Natl. Acad. Sci. USA 95:6448-6453, but similar to the in vitro preparations described by Bitan and Teplow, 2005, Methods Mol. Biol. 299:3-9 and Finder and Glockshuber, 2007, Neurodegener. Dis. 4:13-27. On Western blots (FIG. 2B), the Aβ42 oligomers (O) showed immunosignals at higher molecular weights ranging from 30 to >100 kDa that reacted with the 6E10 and 4G8 antibodies (FIG. 2B, left panel), whereas the non-oligomerized (M) samples showed only low order species. A shorter exposure time revealed a reduction in the number of low order species in the oligomerized samples (FIG. 2B, boxed right panel). Further, oligomerized Aβ42 showed robust punctate binding on dendrites of cultured primary hippocampal neurons (FIG. 2C, arrows showing bound oligomers), consistent with previous reports that Aβ42 oligomers selectively bind to dendritic spines (Lacor et al., 2004, J. Neurosci. 24:10191-10200; Shughrue et al., 2010, Neurobiol. Aging 31: 189-202; Zhao et al., 2010, J. Biol. Chem. 285:7619-7632). In summary, each of these assays confirmed the presence of oligomeric forms in the Aβ42 preparations.

### Aβ42 Oligomer Immunoreactivity

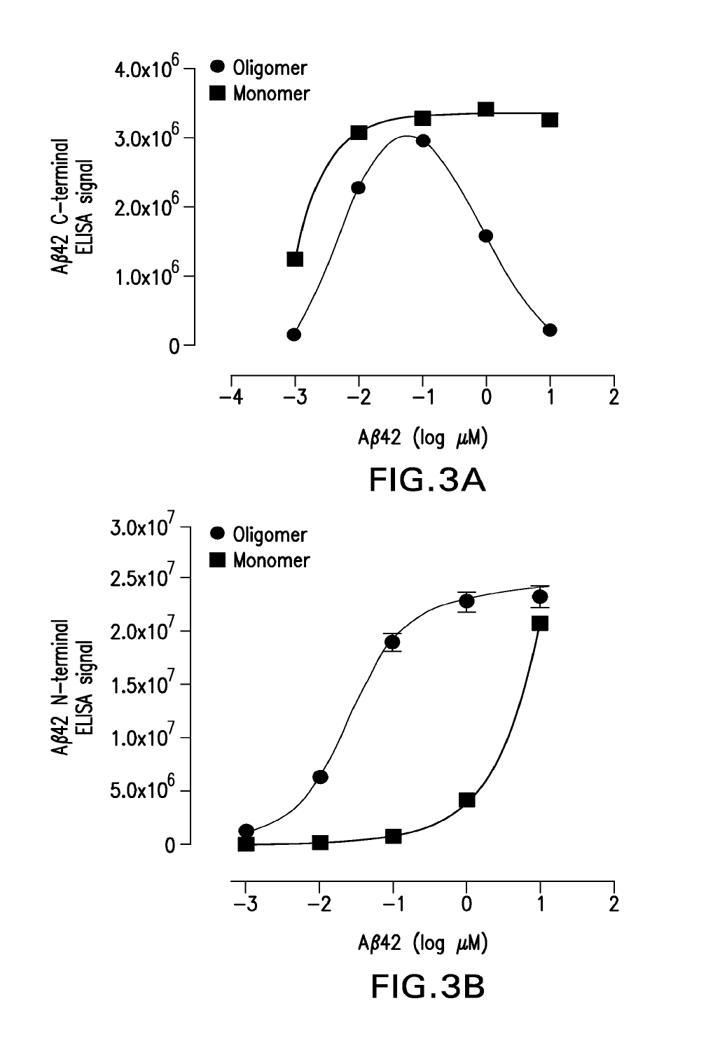

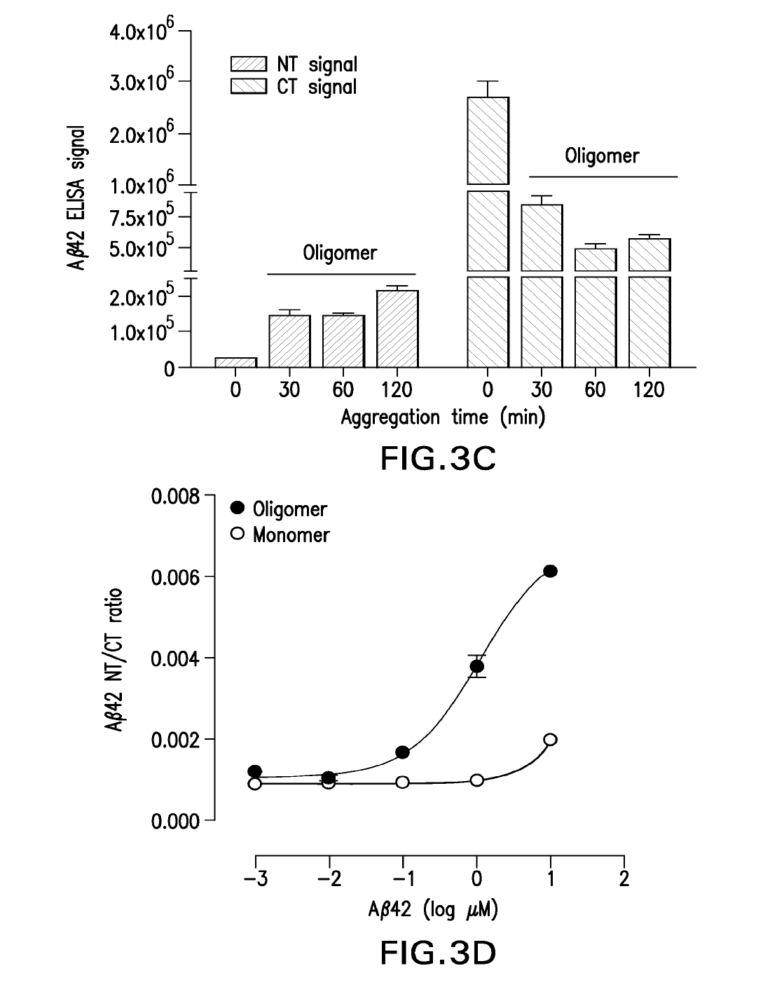

Applicants next measured changes in Aβ42 CT and NT immunoreactivity, i.e. the CT and NT immunosignals, based on the conformational model described above. CT immunoreactivity was detected with a CT specific Aβ42 antibody, 12F4. As shown in FIG. 3A, monomer Aβ42 showed higher CT immunoreactivity than for Aβ42 oligomers at all concentrations tested. For oligomerized Aβ42 (), there was an initial dose-dependent increase in 12F4 immunoreactivity, which was markedly reduced as the Aβ42 concentration increased. In comparison, 12F4 immunoreactivity for the monomer samples (▪) reached a plateau, notwithstanding increases in Aβ42 concentration.

In a parallel experiment, oligomerized () and monomer (control) (▪) Aβ42 peptide was assayed in an NT ELISA, in which 6E10 was used for both capture and detection (FIG. 3B). In contrast to the CT ELISA assay (FIG. 3A), Aβ42 oligomers displayed markedly higher NT immunoreactivity than Aβ42 monomer at most concentrations. The control samples exhibited higher NT immunosignals at 1 μM, indicating the presence of concentration-dependent oligomerization. Unlike prior studies using an NT assay format to detect the presence of Aβ42 oligomers (Fukumoto et al., 2010, FASEB J. 24:2716-2716; Gandy et al., 2010, Ann. Neurol., 2010, 68: 220-230), the increases observed in the NT immunosignal in combination with the concomitant decrease in the CT immunosignal enabled Applicants to detect the formation of such oligomers and to screen for Aβ42 oligomer inhibitors.

A time course experiment herein also showed the inverse changes in the CT and NT immunosignals as early as thirty minutes following initiation of oligomerization at 37° C. (FIG. 3C), indicative of the rapid, spontaneous oligomerization of Aβ42 under these conditions. The results were consistently observed when Aβ42 was oligomerized in different aqueous solutions and buffers, including neurobasal (NB) medium, minimum essential medium (MEM), Dulbecco's modified Eagle's medium (DMEM) and phosphate buffered saline (PBS).

To further confirm the inverse relationship between the NT and CT immunosignals for Aβ42 oligomers, Applicants carried out a multiplex assay in which the detection NT antibody, labeled with Alexa Fluor® 488 (Invitrogen, Carlsbad, Calif.), and the detection CT antibody, labeled with alkaline phosphate (AP) (ABD Serotec, Carlsbad, Calif.), were sequentially applied to the same sets of samples. Consistently, Aβ42 oligomers displayed a higher NT immunosignal and a lower CT immunosignal, leading to a substantially higher NT/CT immunosignal ratio as compared to Aβ42 monomers (FIG. 3D). Taken together, these results demonstrate that the Aβ42 NT immunosignal increased and the Aβ42 CT immunosignal decreased as a result of Aβ42 oligomerization.

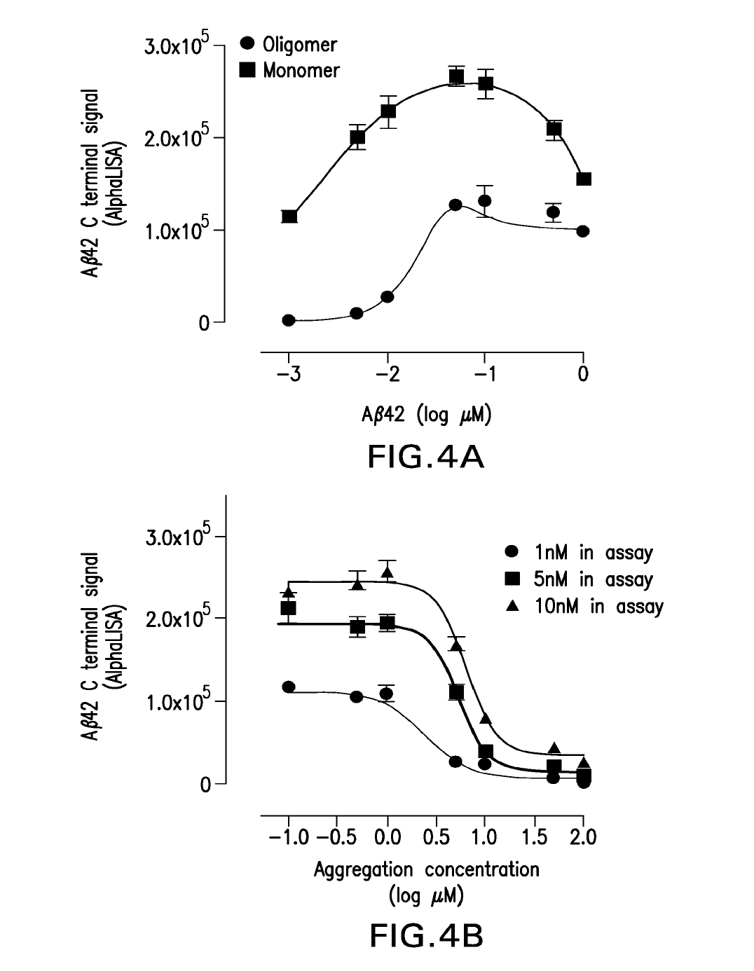

Applicants extended their findings from the CT ELISA assay to an AlphaLISA assay (PerkinElmer, Waltham, Mass.) format, that would enable a high throughput screen with greater efficiency (Eglen et al., 2008). Aβ42 was oligomerized under standard conditions, and the CT immunoreactivity detected following serial dilution. Compared to the ELISA assay, the AlphaLISA assay format generated a significantly higher range in the CT immunosignal between the oligomers and monomer species when measured as low as 1 nM Aβ42 (FIG. 4A). The ability to measure these species even at low concentrations was indicative that the AlphaLISA is a highly sensitive assay for measuring early Aβ42 oligomerization. Further, because the concentration of Aβ42 plays an important role in its oligomerization, Applicants evaluated CT immunoreactivity following oligomerization at Aβ42 concentrations ranging from 100 nM to 100 μM. As shown in FIG. 4B, notwithstanding that the Aβ42 concentration was held steady (1 nM, 5 nM or 10 nM), the oligomers formed from higher Aβ42 concentrations (>5 μM) showed substantially lower CT immunoreactivity than those formed at lower concentrations. This further confirmed that decreases in Aβ42 CT immunoreactivity was a reliable and sensitive surrogate for Aβ42 oligomerization.

### Screen for Aβ42 Oligomer Inhibitors

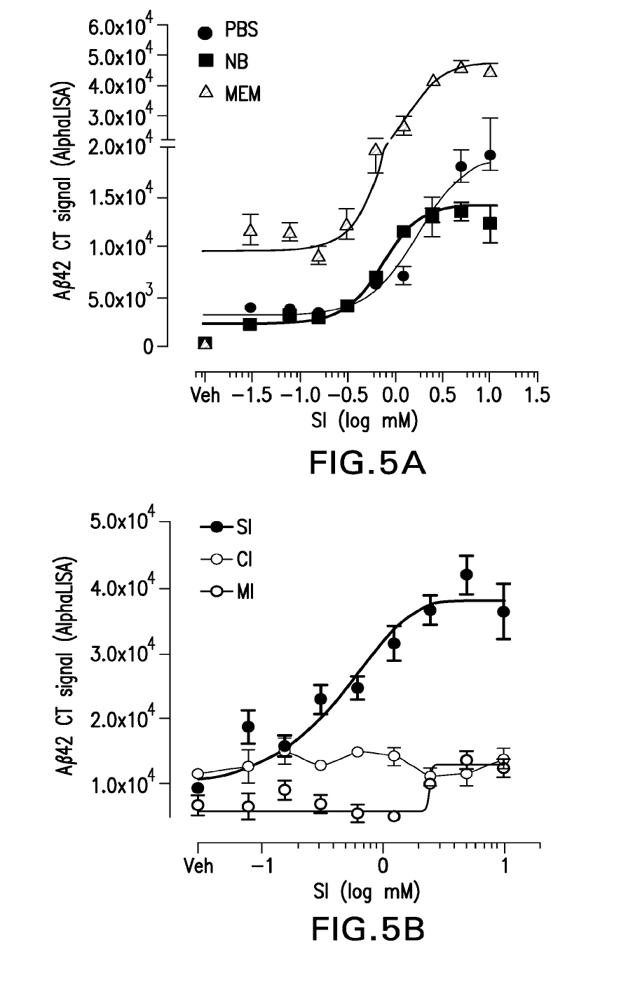

Applicants evaluated the use of the Aβ42 CT and NT immunoassays for the identification of Aβ42 oligomerization inhibitors by testing the effect of known Aβ42 oligomer inhibitors, such as the steroisomer of inositol, scyllo-inositol (SI). In this experiment oligomerization of Aβ42 (1 nM) was induced at 4° C. overnight in the presence or absence of different concentrations of SI. As shown in FIG. 5A, SI produced a dose dependent increase in Aβ42 CT immunoreactivity in different buffers measured by AlphaLISA, whereas the stereoisomers myo-inositol (MI) and chiro-inositol (CI) had no effect on Aβ42 CT immunoreactivity (FIG. 5B). These results are indicative that SI inhibits oligomerization of Aβ42, which follows from the reported findings that SI attenuates the toxic effects attributed to Aβ42 oligomers (McLaurin et al., 2000, J. Biol. Chem. 275:18495-18502; Townsend et al., 2006, Ann. Neurol. 60:668-676). Moreover, in that low concentration, i.e. 1 nM, Aβ42 is unlikely to form fibrils, the results herein suggest that the Aβ42 CT assay of the present invention is sufficiently sensitive to detect early, spontaneous Aβ42 oligomers.

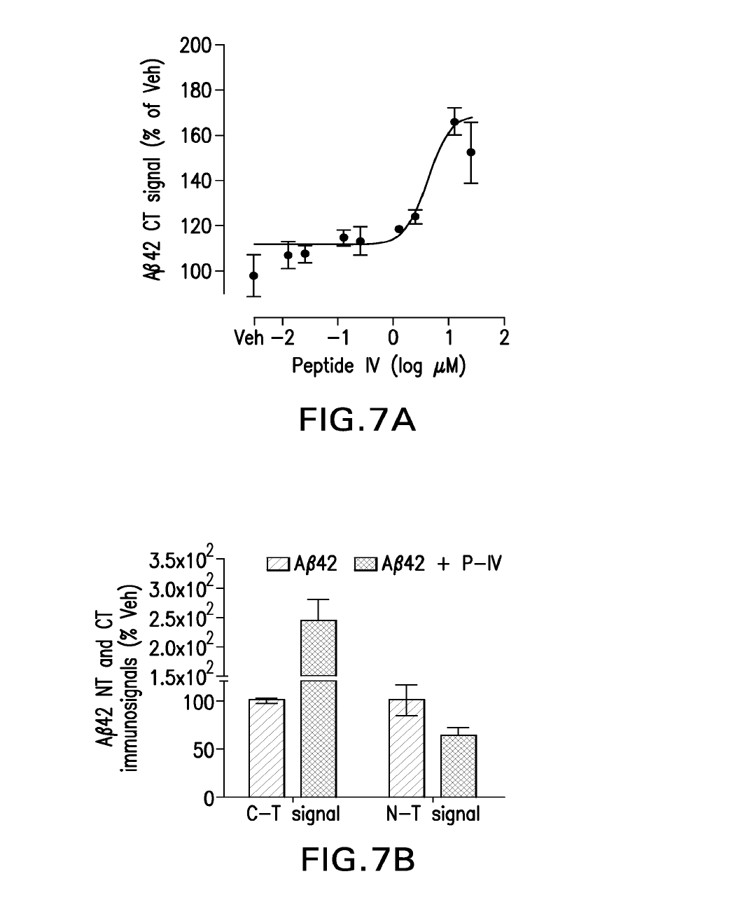

In solution, Aβ42 is metastable, meaning that it is able to maintain an equilibrium between the oligomer and monomer forms of Aβ (Teplow, 2006, Methods Enzymol. 413:20-33; Teplow et al., 2006, Acc. Chem. Res. 39:635-645). As shown in FIGS. 6A and 6B, the addition of SI to pre-formed Aβ42 oligomers resulted in a statistically significant increase (P<0.01), relative to samples assayed in the absence of SI, in CT immunoreactivity and a corresponding significant decrease (P<0.01) in NT immunoreactivity. These results confirm that SI shifted the Aβ42 equilibrium toward monomer. This same effect was also observed with an inhibitory peptide of Aβ42 fibrilogenesis (Peptide IV) (Adessi et al., 2003, J. Biol. Chem. 278:13905-13911). Peptide IV (P-IV) is a commercially available peptide (Calbiochem®, EMD4 Biosciences, Merck KGaA, Darmstadt, Germany), having the sequence Ac-Leu-Pro(N-CH₃)Phe-Phe-Asp-NH2 (SEQ ID NO: 1), that acts as β-sheet breaker, which in turn inhibits Aβ42 oligomerization. Peptide IV generated a dose-depended increase in CT immunoreactivity (FIG. 7A) and a corresponding significant decrease (P<0.01) in the NT immunosignal (FIG. 7B). Taken together, these results demonstrate that the Aβ42 CT and NT AlphaLISA assays were effective in evaluating compounds that affect oligomerization of Aβ42, i.e., Aβ42 oligomer inhibitors.

### Aβ42 Oligomerization Measured by Dynamic Light Scattering

To validate the results of the CT and NT immunoassays, Applicants used dynamic light scattering (DLS), which measures changes in particle size, to demonstrate the inhibitory effect of scyllo-inositol (SI) on Aβ42 oligomer formation. Aβ42 (50 μM) showed time-dependent oligomerization with the average radius increasing from 48 nm, at thirty minutes post incubation, to 61 nm, at seven hours post-incubation (Table 1). At thirty minutes post-incubation, the Aβ42 peptide showed a major peak evident between 3 nm and 8 nm. With increasing oligomerization time, the peak shifted to the right, forming two roughly equal peaks distributed between 10 nm and 100 nm (FIG. 8). The percent polydispersity (% PD) and the sum of squares (SOS) are two parameters uses to represent the uniformity and range of size, shape and mass characteristics of particles in solution. The higher the % Pd and SOS, the more heterogeneous the particles are in size and shape.

TABLE 1

| Time | Temperature | Norm Intensity | Average | | |
|---|---|---|---|---|---|
| (hours) | (° C.) | (Cnt/s) | Radius (nm) | % Pd | SOS |
| 0.5 | 25 | 2413450.0 | 48.1 | 23.8 | 14.3 |
| 7.0 | 25 | 2486300.0 | 61.0 | 23.9 | 2.4 |

Table 2 shows the effect of SI on Aβ42 oligomerization. In the absence of SI, Aβ42 (40 μM) formed two major peaks. The average radius of peak 1 was 5.2 nm and composed of 56.8% mass, whereas peak 2 showed an average radius of 21.5 that occupied 66% mass. In the presence of SI, the amount of peak 1 increased to 76% mass, with the average radius reducing to 2.5 nm. Although the radius of peak 2 remained unchanged, the amount was reduced to 38% mass. These results indicate that Aβ42 oligomerization was inhibited in the presence of SI, consistent with the results observed with Aβ42 CT immunoassays.

TABLE 2

| | | Temperature | Radius | MW-R | % | % |
|---|---|---|---|---|---|---|
| | Item | (° C.) | (nm) | (kDa) | Intensity | Mass |
| Peak 1 | Ab42 | 25 | 5.2 | 41.35 | 1.55 | 56.8 |
| | Ab42/SI | 25 | 2.5 | 11.6 | 0.7 | 72.93 |
| Peak 2 | Ab42 | 25 | 21.5 | 530.2 | 98.33 | 66.15 |
| | Ab42/SI | 25 | 21.4 | 527.1 | 96.88 | 38.75 |

### Aβ42 CT AlphaLISA Assay as High Throughput Screen

Applicants automated the Aβ42 C-terminal (CT) AlphaLISA assay for high throughput screen (HTS) using a Echo555 (Labcyte, Sunnyvale, Calif.) and a Bravo automatic liquid handler (Agilent Technologies, Santa Clara, Calif.). The automated assay results confirmed that loss of CT immunoreactivity, i.e. loss of CT immunosignal, correlated with Aβ42 oligomerization. Applicants evaluated about 2,000 compounds from different structural classes for their effects on Aβ42 oligomerization. A single concentration of 10 μM was used for the initial screen, and a threshold of 3-fold standard deviation (3SD) of the oligomerized samples in the absence of compound (DMSO vehicle controls) was selected as the cutoff for inhibitor hits. Compounds showing increases in the Aβ42 CT immunosignal above this threshold were selected and tested for dose response effects in a second round screen. About 4% of the compounds showed dose-dependent increase in Aβ42 CT immunoreactivity. Representative results are shown in FIGS. 9A-9D. In the primary screen (FIGS. 9A and 9C), DMSO vehicle was used as a control for the oligomerization baseline (solid line). Three times the standard deviation (3× SD) was used as a cutoff (dotted line) for oligomerization inhibition. Compounds generating a CT immunosignal above the 3× SD cutoff were deemed to be an oligomerization inhibitor hit.

In both Class I and Class II compounds, classes that represented compounds having distinct chemical structures, the majority of compounds in each group showed CT AlphaLISA immunosignals similar to or below the DMSO (control) baseline, indicating no effect on inhibition Aβ42 oligomerization. A small proportion of compounds generated CT immunosignals slightly higher than the DMSO control, but still below the 3× SD cutoff. Only a small number of compounds had a CT immunosignal above the 3× SD cutoff, hits which suggested their potential to inhibit Aβ42 oligomerization. To confirm their oligomerization inhibitory effect, compounds producing signals above 3× SD were tested again in a dose-dependent assay. FIGS. 9B and 9D represent the increase in the CT immunosignal observed for example hits from each structural class that were evaluated in a dose dependent manner.

Applicants also assessed the quality of this assay using Z′ factors, commonly used to quantify the suitability of an assay for use in a full-scale, high-throughput screen (HTS). Calculation of the screen window coefficient (Zhang et al., 1999, J. Biomol. Screen. 4:67-73) for a positive tool compound generated an average Z′ factor of 0.64, assuring high confidence of the assay. The Z-factor is computed from four parameters, the means and standard deviations of both the positive (p) and negative (n) controls (μp,σp, and μn,σn), and is defined as:

$$Zfactor = 1 - \frac{3 \times ( {σ}_{p} + {σ}_{n} )}{\vert {μ}_{p} - {μ}_{n} \vert} .$$

An alternative but equivalent definition of Z-factor is calculated from the Sum of Standard Deviations (SSD=σp+σn) divided by the range of the assay (R=|μp−μn|):

$$Zfactor = 1 - 3 \times \frac{SSD}{R} .$$

Assays having Z-factors in the following ranges are generally evaluated as follows:

| | |
|---|---|
| 1.0 | Ideal. Z-factors can never actually be greater than or equal to 1.0 |
| 0.5-1.0 | Excellent. Note: for σp = σn, 0.5 is equivalent to a |
| | separation of 12 standard deviations between μp and μn. |
| 0.0-0.5 | Marginal. |
| <0.0 | No value. Note: values less than 0.0 indicate that the signal from |
| | the positive and negative controls overlap. |

Because an increase CT immunosignal from the AlphaLISA were used to determine a compound's ability to inhibit oligomerization, it was speculated that the positive AlphaLISA signal could be the result of the compound's non-specific interaction with the donor and acceptor beads leading to a false positive hit. To exclude this possibility, the compounds were tested in the same assay without the presence of Aβ42. If the assay signal is specific to the CT of Aβ42, conducting the assay in the absence of Aβ42 would result in a negative immuno signal readout. Indeed, most compounds showed negative results in the absence of Aβ42 (data not shown).

Additionally, because Aβ42 oligomerization was carried out in a polypropylene plate at a low nM concentration and then transferred to a second plate for the assay reaction, a concern was raised that any soluble Aβ42 might stick to the oligomerization plate and interfere with the assay. Prevention of non-specific binding of Aβ42 to the first plate would result in a higher amount of Aβ42 being transferred to the assay plate, which in turn would lead to increased CT signals. Thus, a false positive result could be generated by a compound that prevented Aβ42 sticking to the plate rather than inhibition of oligomerization. To address this concern, Applicants used an NT (4G8-6E10) ELISA (Example 7) to measure the total amount of Aβ42 transferred from the first plate after oligomerization in the absence or presence of a Compound C, a known Aβ42 oligomer. If Compound C caused an increase in the CT immunosignal by preventing Aβ42 sticking to the plate, a higher 4G8-6E10 signal would be obtained compared with the non-compound control. Simultaneously, a CT (4G8-12F4) ELISA was performed, the results of which reflect the oligomerization state of the transferred Aβ42 peptides (FIG. 10). There was no apparent difference in the NT (6E10) immunoreactivity among samples with and without compound (FIG. 10, 4G8-6E10 pair). However, the CT immunosignal detected by the 4G8-12F4 antibody pair was substantially higher in samples treated with the compound (FIG. 10). These results indicate that while this compound did not affect the total amount of Aβ42 transferred from the oligomerization plate, it inhibited oligomerization and resulted in the presence of more Aβ42 monomer. Taken together, the results demonstrated that the automated Aβ42 CT AlphaLISA was a sensitive, reproducible, and robust assay for HTS of small molecule inhibitors of Aβ42 oligomerization.

### EXAMPLES

The following abbreviations are used herein: BSA: bovine serum albumin; CT: C-terminal; NT: N-terminal; HTS: high-throughput screen; DMSO: dimethyl sulfoxide; PBS: phosphate buffered saline; NB: neurobasal culture medium; MEM: Minimum essential medium; HFIP: hexafluoroisopropanol; SI: scyllo-nositol.

### Example 1

### Preparation of Aβ42 Oligomers

Nonbinding polypropylene tubes were used for handling A131-42. Synthetic human Aβ1-42, purchased from American Peptide Company (Sunnyvale, Calif.), was dissolved in 1,1,1,3,3,3,-hexafluoro-2-propanol (HFIP) (Sigma-Aldrich Corp., St. Louis, Mo.) to 1 mM and incubated at room temperature for 30 minutes to remove secondary structures of the peptide. Following aspiration of HFIP, the peptide was lyophilized in a vacuum concentrator (SpeedVac®, Thermo-Fisher Scientific, Waltham, Mass.) and stored at −80° C. until use. The HFIP dry film was dissolved in dimethyl sulfoxide (DMSO) (Sigma-Aldrich Corp., St. Louis, Mo.) to 1 mM to form a stock solution, which was aliquoted into small quantities (100 μl) and stored at −80° C. until used. To prepare the Aβ42 oligomers, 1 mM of the Aβ42 DMSO stock solution was diluted in 10× series in nonbinding polypropylene microtubes to 100 μM, 10 μM, and 1 μM, in a buffer (e.g. PBS) or in medium (e.g. Neurobasal).

To generate pre-fibrillar oligomers, a diluted Aβ42 solution (e.g. 10 μM or 100 μM) was incubated in a microtube on an Eppendorf Thermomixer® (Eppendorf, Hamburg, Germany) at 37° C. without shaking for 60 minutes (incubation time was varied from 30 minutes to 180 minutes to test different degrees of oligomerization). Oliomerization of Aβ42 was verified by SDS-PAGE and AFM. The oligomerized solution was again serially diluted to 50 μM, 10 μM, 5 μM, 1 μM, 100 nM, 50 nM, 10 nM, 5 nM and 1 nM before use in an immunoreactive detection assay. For the non-oligomer control, an aliquot (1 mM) of the same Aβ42 DMSO stock solution was diluted to the same concentrations as the oligomerized samples immediately prior to assay.

### Example 2

### Aβ42 Inhibitor Assays

A. Metastability

The effect of an Aβ oligomer inhibitor on the metastability of Aβ42 oligomers prepared according Example 1 was evaluated as follows. A sample of the 1 mM Aβ42 DMSO stock was diluted to 10 μM or 100 μM in PBS or Neurobasal (e.g., 10 μl of 1 mM Aβ42 DMSO stock to make 1 ml 10 μM solution or 100 μl A(342 DMSO stock to make 1 ml 100 μM solution). After incubation at 37° C., the diluted oligomer solutions were mixed with scyllo-inositol (SI), a naturally occurring plant sugar alcohol found most abundantly in the coconut palm, to make a final concentrations of 1 μM or 10 μM Aβ42 containing 10 mM SI. The non-compound control oligomer sample contained no SI. The mixtures were incubated on ice for two to three hours to allow establishment of new equilibrium (metastability) among Aβ42 species. The stabilized mixtures were then utilized in the immunoreactive assays.

B. Early Oligomerization

To test a compound's effect on inhibition of early oligomerization, SI, or a different testing compound, was mixed with the Aβ42 solution to make solutions with final concentrations of SI from 0.1 μM to 10 mM (0.1 μM, 1 μM, 10 μM, 100 μM, 1 mM, and 10 mM) and Aβ42 from 1 nM to 10 μM (1 nM, 10 nM, 100 nM, 1 μM and 10 μM). For example, a 10 μM Aβ42 solution contained, respectively, 0 μM, 0.1 μM, 1 μM, 10 μM, 100 μM, 1 mM and 10 mM SI. The same SI concentration series was applied to other concentrations of the Aβ42 solution indicated above. The mixtures were either incubated at 4° C. overnight or room temperature for two to three hours, before being used in the immunoreactive assay.

### Example 3

### Aβ42 C-Terminal (CT) Oligomer ELISA

An Aβ42 C-terminal (CT) oligomer assay was performed using an ELISA format as follows. Briefly, a 96-well black OptiPlate™ (PerkinElmer, Waltham, Mass.) was coated with (100 μl/well) 5 μg/ml of a capture antibody, 6E10, an antibody that recognizes an epitope in the N-terminal (NT) region of Aβ, prepared in a sodium bicarbonate buffer (Sigma-Aldrich Corp., St. Louis, Mo.) and incubated at 4° C. overnight. The plate was then blocked with 5% bovine serum BSA (Sigma-Aldrich Corp., St. Louis, Mo.) made in phosphate buffered saline containing 0.05% Tween 20 (Sigma-Aldrich Corp., St. Louis, Mo.) (PBST) for 10 to 12 hours. After rinsing the plate once with 1× PBST, Aβ42 oligomer or monomer samples were added to the plate (100 μl/well) and incubated at 4° C. overnight. After removal of unbound samples, the plate was washed with 1× PBST for six times. The plate was then incubated with 100 μl of a detection antibody, 12F4, an antibody that recognizes an epitope in the CT region of Aβ42, conjugated to alkaline phosphate (AP) (1:5,000), at room temperature for two hours. The unbound antibody solution was removed and the plate was washed with PBST six times. The plate was then reacted with an alkaline phosphatase (AP) chemiluminescent substrate (CDP-Star®, Applied Biosystems by Life Technology Corp., Carlsbad, Calif.), at room temperature for thirty minutes. The immunoreactive signals were read with a multiplate reader (EnVision®, PerkinElmer, Waltham, Mass.). The presence of Aβ42 oligomers was determined by a corresponding decrease in the CT immunosignal. The extent of oligomerization is shown to be inversely correlated with the magnitude of the CT immunoreactivity, i.e., the higher the oligomerization, the lower the CT immunosignal. Values for the ELISA assays were graphed and analyzed with Prism GraphPad software.

### Example 4

### Aβ42 C-Terminal (CT) Oligomer AlphaLISA Assay

Similarly, the presence of Aβ42 oligomers was detected using an Aβ42 C-terminal (CT) AlphaLISA assay (Eglen et al., 2008, Curr. Chem. Genomics, 1:2-10), a bead based proximity assay, based upon an oxygen channeling technology. The assay was carried out in a 384 well plate using an AlphaLISA® Human Amyloid β 1-□42 Research Immunoassay Kit (PerkinElmer, Waltham, Mass.) according to the manufacturer's directions. To make a 2.5× AlphaLISA acceptor bead and biotinylated anti-Aβ42 antibody mixture, 5.5 μl acceptor beads conjugated to anti-Aβ42 (12F4) and 5.5 μl biotin-Aβ42 antibody (binds to a epitope away from the CT of Aβ42) were added to 1,089 μl 1S AlphaLISA buffer and mixed by brief vortexing. To each well of a 384-well plate, 8 μl of the mix and 2 μl of an Aβ42 sample were added, followed by gentle tapping of the plate to mix the solutions. The plate was incubated at room temperature for one hour. The streptavidin donor bead solution was made in a dark room under safety light by mixing 20.1 μl of streptavidin donor beads with 1,279 μl 1× AlphaLISA buffer. The streptavidin donor bead solution, which binds to the biotinylated anti-Aβ42 antibody, was then added (10 μl/well) to the plate, sealed with an adhesive aluminum membrane, and incubated at room temperature for 30 minutes. Incubation brought the donor and acceptor beads into close proximity. An immunoreactive signal was generated when the donor bead released a singlet oxygen that, when excited at 680nm, was transferred to the acceptor bead, resulting in a light emission at 610 nm. The immunoreactive signals were read with a multilabel plate reader (EnVision®, PerkinElmer, Waltham, Mass.). The presence of Aβ42 oligomers was determined by a corresponding decrease in the C-terminal immunosignal. Values for the AlphaLISA assays were graphed and analyzed with Prism GraphPad software. Similar to signals generated with ELISA, the extent of Aβ42 oligomerization is inversely correlated with the magnitude of the CT immunosignal in AlphaLISA. Values for the ELISA assays were graphed and analyzed with Prism GraphPad software.

### Example 5

### Aβ42 N-Terminal (NT) Oligomer ELISA

An Aβ42 N-terminal (NT) oligomer ELISA was performed by adopting a 6E10-6E10 ELISA originally developed in house at Merck and also reported by others (Gandy et al., 2010, Ann. Neurol. 68: 220-230; Xia et al., 2009, Arch. Neurol. 66:190-199). Briefly a 96-well black OptiPlate™ (PerkinElmer, Waltham, Mass.) was coated with 5 μg/ml 6E10 antibody in carbonate/bicarbonate buffer pH 9.5 and incubated overnight at 4° C. The 6E10 antibody recognizes an epitope in the NT region of Aβ. The plate was then blocked with 200 μl/well 5% BSA-PBST-overnight at 4° C. As illustrated in FIG. 1C, Aβ42 oligomer and/or monomer samples (100 μl/well) were added to the plate and incubated at 4° C. overnight. The unbound samples were removed and plate washed with 1× PBST for 6 times. The plate was then incubated with 100 μl of a detection antibody (1:5,000), 6E10, identical to the capture antibody, but conjugated to AP, at room temperature for two hours. After 6 washes with 1× PBS, the plate, coated with 100 μl/well an alkaline phosphatase (AP) chemiluminescent substrate (CDP-Star,® Applied Biosystems by Life Technology Corp., Carlsbad, Calif.), was reacted at room temperature for thirty minutes. The immunoreactive signals were read with a multilabel plate reader (EnVision®, PerkinElmer, Waltham, Mass.). The presence of Aβ42 oligomers was determined by a corresponding increase in the NT immunosignal. Values for the ELISA assays were graphed and analyzed with Prism GraphPad software. As illustrated in FIG. 1C, because the capture and detection antibodies are identical, only oligomer species that were bound by at least two IgG molecules of 6E10 were detected. The extent of oligomerization was correlated with the magnitude of the NT immunosignal; greater degrees of oligomerization result in higher NT immunosignals until the reaction reaches the saturation of the 6E10 antibody.

### Example 6

### CT AlphaLISA for HTS of Aβ42 Oligomer Inhibitors

For high throughput compound screening (HTS), the Aβ42 CT AlphaLISA assay described above (Example 4) was miniaturized and automated as follows. A 10 mM compound source plate was prepared by adding 8 μl of a test compound (10 mM) to a 384-well low dead volume (LDV) plate (Labcyte, Sunnyvale, Calif.). Using an acoustic liquid handler (ECHO®, Labcyte, Sunnyvale, Calif.), 250 nl of the 10 mM compound was transferred from the compound source plate to an polypropylene round bottom assay plate (assay plate #1) (Costar, Lowell, Mass.), to a compound final concentration of 100 μM. Next, an Aβ42 source plate was prepared by diluting 1 mM Aβ42 DMSO stock (as above in Example 1) to 600 nM in 100% DMSO (Sigma-Aldrich, St. Louis, Mo.) in a maximum recovery 1.7 ml microfuge tube (Axygen, Union City, Calif.), from which 8 μl of the 600 nM Aβ42 was manually pipetted into a 384 well LDV plate (Labcyte, Sunnyvale, Calif.). An acoustic liquid handler (ECHO Labcyte, Sunnyvale, Calif.) was used to transfer 50 nl of the Aβ42 from the Aβ42 source plate to assay plate #1 to mix with the added compound as described above. Aβ42 final concentration in each well was 1.5 nM. A liquid handler (Bravo, Agilent Technologies, Santa Clara, Calif.) was used to add 19.7 μl PBS and bring the final assay volume in each well of assay plate #1 to 20 μl. The plate was then sealed with foil adhesive and incubated for 4 hours at 4° C. for oligomerization.

To perform a HTS for Aβ42 inhibitors using an AlphaLISA CT Aβ42 assay, 8 μl of the AlphaLISA acceptor bead and biotinylated anti-Aβ42 antibody mix (see Example 4) was dispensed to a 384 well polystyrene assay plate (assay plate #2) using a liquid handler (Bravo, Agilent Technologies, Santa Clara, Calif.). The plate was sealed with foil adhesive and incubated for one hour at room temperature. Following incubation, 10 μl of the streptavidin donor bead was added to assay plate #2 with the liquid handler (Bravo, Agilent Technologies, Santa Clara, Calif.). The plate was again sealed and incubated for 30 minutes at room temperature before being read on a multiplate reader (EnVision®, PerkinElmer, Waltham, Mass.).

### Example 7

### Total Aβ42 ELISA

In the HTS compound screen assay, Aβ42 oligomerization was performed in a polypropylene plate (assay plate #1) and the AlphaLISA was performed in a polystyrene assay plate (assay plate #2), after sample was transferred from the first plate to preclude the loss of Aβ42 due to adherence to the plastic plate. This assay validated that the observed reduction in the Aβ42 NT immunosignal resulted from Aβ42 oligomerization and not from loss of Aβ42 due to peptide sticking to the plastic plate. Conversely, the observed increase in Aβ42 NT immunosignal was attributed to the test compound inhibiting oligomerization and not because the compound prevented Aβ42 from sticking to the plate. If the test compound did not affect Aβ42 sticking to the plastic plate, then the total amount of Aβ42 transferred between plates would not change regardless of the presence or absence of the compound during oligomerization.

The 1 mM Aβ42 DMSO stock was diluted in 10× series to 1.5 nM with PBS and incubated in a 384-well LDV plate (see Example 6) for 4 hours at 4° C. in the presence and absence of a test compound (Compound C), that was shown to inhibit Aβ42 oligomerization in the screen described in Example 6. Upon oligomerization, 100 μl/well of the Aβ42-compound mixture was transferred to a high binding 96-well microplate coated with 5 μg/ml of the monoclonal Aβ antibody, 4G8, which recognizes an epitope corresponding to amino acid positions 17-24 of Aβ42, and blocked with 5% BSA-PBST. The samples were incubated with the plate at 4° C. overnight. Following six washes with PBST, the NT antibody, 6E10, conjugated with AP, was added to the plate (1:5000;100 μl/well) at room temperature for 2 hours. Simultaneously, the CT antibody, 12F4, conjugated with AP, was added (1:3000, 100 μl/well) to a duplicate set of samples on the same plate and incubated at room temperature for 2 hours. After six washes with PBST, the plate was incubated with an AP chemiluminescent substrate (CDP-Star®, Applied Biosystems by Life Technology Corp., Carlsbad, Calif.) at room temperature for thirty minutes, followed by reading the plate on a multiplate reader (EnVision®, PerkinElmer, Waltham, Mass.). Values for the ELISA assays were graphed and analyzed with Prism GraphPad software. Because the capture antibody and the detection antibody in the 4G8-6E10 pair recognize different epitopes on Aβ42, the NT was available for 6E10 binding regardless of whether Aβ42 was in monomer or oligomeric forms. Thus, the ELISA immunosignal for the 4G8-6E10 pair reflected the total amount of Aβ42 peptide, while the 4G8-12F4 pair reflected the immunosignal decrease when Aβ42 oligomerizes.

### Example 8

### Multiplex ELISA to Detect Oligomerization

One of ordinary skill in the art would appreciate and recognize that an assay that can simultaneously detect N-terminal (NT) and C-terminal (CT) immunosignals in the same well of a reaction plate, using NT and CT Aβ42 antibodies labeled with different fluorescent dyes, will reduce cross-well sample handling error. Briefly, a 96-well black OptiPlate™ (PerkinElmer, Waltham, Mass.) is coated with 5 μg/ml 6E10 antibody in carbonate/bicarbonate buffer pH 9.5, and blocked with 5% BSA-PBST as described in Example 3. Oligomer or monomer Aβ42 samples (at similar concentrations described in Example 3 and Example 7) are added (100 μl/well) to the plate at 4° C. overnight to allow binding. After washing the plate at least six times with PBST, the NT antibody, 6E10, conjugated with Alexa Fluor® 488 (Molecular Probes, a subsidiary of Invitrogen, Carlsbad, Calif.) and the CT antibody, 12F4, conjugated with Alexa Fluor® 647 (Invitrogen, Carlsbad, Calif.) are added to the plate (1:3000, 100 μl well) and incubated at room temperature for 1 to 2 hours. The conjugating fluorescent dyes used to label each antibody can vary and can be used to distinguish the antibodies by detection with separate filters in a reading apparatus, such as, a multiplate reader (EnVision®, PerkinElmer, Waltham, Mass.). After washing the plate at least 6 times with PBST, the plate is read with a multiplate reader (EnVision®, PerkinElmer, Waltham, Mass.) using a built-in fluorescent protocol for maximal emission of 519 nm and 665 nm, respectively. The ratio of NT to CT signals is calculated and data analyzed with GraphPad software.

### Example 9

### Fluorescent-Luminescent Multiplex Oligomerization ELISA

This assay can also be used to simultaneously detect NT and CT immunosignals (Example 8) to avoid potential between-well fluorescent crosstalk. The procedure is as follows. A 96-well black OptiPlate™ (PerkinElmer, Waltham, Mass.) is coated with 5 μg/ml 6E10 antibody in carbonate/bicarbonate buffer pH 9.5, and blocked with 5% BSA-PBST (see, Examples 3, 7, and 8). Oligomer or monomer Aβ42 samples (at concentrations similar to those described in Examples 3 and 7) are added (100 μl/well) to the plate at 4° C. overnight to allow binding. After washing the plate at least 6 times with PBST, a mix solution of the NT antibody, 6E10, conjugated with Alexa Fluor® 488 (Molecular Probes, a subsidiary of Invitrogen, Carlsbad, Calif.) (1:3000) and a CT antibody, 12F4, conjugated with AP (1:3000), are added to the plate and incubated at room temperature for 1 to 2 hours. After washing at least 6 times with PBST, the plate is read on a multiplate reader (EnVision®, PerkinElmer, Waltham, Mass.) using a built-in fluorescent protocol suitable for detecting the Alexa Fluor® 488 signal. The plate is then incubated with an AP chemiluminescent substrate (CDP-Star®, Applied Biosystems by Life Technology Corp., Carlsbad, Calif.) at room temperature for thirty minutes, followed by reading on a multiplate reader (EnVision®, PerkinElmer, Waltham, Mass.) using a luminescent protocol. The NT to CT ratio from the same well is calculated and data is analyzed with GraphPad software.

### Example 10

### Atomic Force Microscopy

Atomic force microscopy, which allows for direct observation of the morphology and size of the Aβ42 oligomers prepared with the protocols herein, was performed to validate oligomerization of Aβ42. The assay was carried out using known methods (see, for example, Lambert et al., 1998, Proc. Natl. Acad. Sci. USA, 95:6448-6453; Stine, Jr. et al., 1996, J. Protein Chem. 15:193-203). A MultiMode atomic force microscope (Digital Instruments/Veeco Metrology, Santa Barbara, Calif.), controlled by a NanoScope IIIa with NanoScope Extender electronics and Q-Control (nanoAnalytics, Münster, Germany) and using the NanoScope operating software version 5.31r1, was used to acquire the data images. Nanoscope offline software was used to render the data after zero-order flattening of the image background. SPIP software version 5.1.0 (Image Metrology A/S, Hørsholm Denmark) was used to perform the particle analyses after applying a Gaussian smoothing function (kernel size=7, 1 standard deviation) to the data. The average z-height and diameter of >50 globules from a one micron area on the mica were determined using a watershed—dispersed features algorithm with a smoothing filter size of 6 pixels.

### Example 11

### SDS-PAGE and Western Blots

SDS-PAGE was used to separate Aβ42 oligomer species. Because different species migrate to positions corresponding to their molecular weight, i.e., according to the size of the oligomers, this assay provided an approximation of the species present, such as, trimers, tetramers, hexamers, etc. Oligomerized Aβ42 samples and controls were treated with non-reducing SDS sample buffer containing 0.05% SDS and resolved on 4-20% precasted Tris-Glycine polyacrylamide gels (Invitrogen, Carlsbad, Calif.), and transferred to nitrocellulose membrane using an iBlot dry blotting system (Invitrogen, Carlsbad, Calif.). Aβ42 immunosignals were detected with a combination of biotin-6E10 and biotin-4G8, followed by subsequent reaction with the combination of streptavin-HRP and anti-mouse HRP. The immunosignal was detected by reacting with a chemiluminescent substrate, such as, SuperSignal West Femto Substrate (Thermo Fisher Scientific, Rockford, Ill.), followed by development of the immunosignal on an X-ray film with a film processor. The subsequent immunosignal on the film was acquired with a densitomic scanner and the image was processed with Adobe PhotoShop software (Adobe Systems Inc, San Jose, Calif.).

### Example 12

### Dynamic Light Scattering

Dynamic light scattering (DLS), also known as also known as quasi-elastic laser light scattering, offered another methodology to determine Aβ42 oligomerization by measuring the size distribution profile and shape of particles in solution. Because DLS does not involve immunoreactions, it provided the advantages of high throughput, minimal reagent requirements, simple reaction steps, and label-free measurement of the change in oligomer size and shape in the presence or absence of an inhibitor compound over time.

Sample preparation for the DLS assay was performed in a bio-safety cabinet. All solutions and reagents were pre-filtered with a 0.1 μm Whatman filter (Whatman, Piscataway, N.J.). Aβ42 (100 μM) made in PBS from the 1 mM DMSO stock (Example 1) was filtered with a 0.2 μm filter (Whatman, Piscataway, N.J.) and diluted to 50 μM to 10 μM with PBS or water. The samples were added to the DLS plate (50 μl/well) in the presence or absence of compounds and incubated at room temperature for seven to eight hours. The plate was briefly centrifuged (1 minute at 3000 rpm) and placed in the DynaPro DLS plate reader (Wyatt Technology, Dernbach, Germany), in which different parameters (normalized intensity, hydrodynamic radius, molecular weight, relative molecular mass, percent polydispersity, and sum of square) of the Aβ42 oligomer samples were measured, and analyzed with Dynamics 7.0.0 software (Wyatt Technology, Dernbach, Germany).

### Example 13

### Aβ42 Oligomer Binding on Primary Neurons

Binding to dendritic spines in cultured hippocampal neurons is a characteristic of Aβ42 oligomers, but it has also been observed with other types of soluble Aβ42 oligomers, such as ADDLs (Lacor et al., 2004, J. Neurosci. 24:10191-10200). Neuronal binding studies were carried out to determine whether the Aβ42 oligomers prepared herein exhibited typical neuronal dendritic binding. Aβ42 oligomer binding to neurons would be indicative of potential toxicity to synaptic structures.

Binding of Aβ42 oligomers to primary hippocampal neurons was performed with primary hippocampal cultures prepared from E18 rat brains as described previously (Zhao et al., 2010, J. Biol. Chem. 285:7619-7632). Briefly, oligomerized Aβ42 samples (500 nM) were applied to hippocampal neurons at day 21 in vitro (DIV) and incubated for fifteen minutes. Neurons were fixed with 4% formaldehyde/4% sucrose made in lx PBS at room temperature for ten minutes. After permeabilization and blockage with 15% normal goat serum, Aβ42 oligomer binding was detected with an NT Aβ antibody, 6E10, which was incubated with cells at 4° C. overnight, followed by incubation with a secondary anti-mouse IgG conjugated with Alexa Fluor® 555 dye (Molecular Probes, a subsidiary of Invitrogen, Carlsbad, Calif.). The fluorescent labeled images were acquired with a Nikon epifluorescent microscope.

Data analysis: Aβ42 NT and CT ELISA and CT AlphLISA raw data were acquired on oligomerization with a plate reader (EnVision®, PerkinElmer, Waltham, Mass.) and were analyzed and plotted with GraphPad software. Concentration dependent effects of Aβ42 oligomerization and compound effects were analyzed with nonlinear regression (curve fit). Atomic force microscopy data was acquired with NanoScope operating software version 5.31r1 and analyzed with SPIP software version 5.1.0 (Image Metrology A/S, Hørsholm Denmark) following rendering the data with Nanoscope offline software after zero-order flattening of the image background. Dynamic light scattering data was analyzed with Dynamics 7.0.0 software (Wyatt Technology, Dernbach, Germany). HTS data for Aβ42 inhibitors was analyzed and curve fit performed with Merck automated data analysis (ADA) system.

What is claimed:

1. An Aβ42 C-terminal (CT) oligomer immunoassay to detect Aβ42 oligomers comprising the use of a capture antibody, that recognizes an epitope in the N-terminal (NT) region of Aβ42, and an alkaline phosphatase (AP) conjugated detection antibody, that recognizes an epitope in the C-terminal regional of Aβ42, that are reacted in the presence of an AP chemiluminescent substrate to produce a CT immunosignal, wherein said CT immunosignal will decrease, relative to the CT immunosignal generated in the absence of Aβ42 oligomers, when Aβ42 oligomers are detected.

2. An assay of claim 1 wherein the capture and detection antibodies are 6E10 and 12F4, respectively.

3. An Aβ42 C-terminal (CT) oligomer bead based proximity immunoassay to detect Aβ42 oligomers comprising:
a. incubating simultaneously together to form a reaction mixture,
i. a strepavidin coated donor bead, that binds to a biotinylated Aβ antibody that recognizes an epitope both in Aβ42 and Aβ40;
ii. an acceptor bead conjugated to a second antibody that recognizes an epitope at the C-terminal region of Aβ42; and
iii. one or more samples of Aβ42;
b. incubating the reaction mixture with a second streptavidin donor bead that binds to said biontinylated Aβ antibody to produce a CT immunosignal; and
c. detecting said CT immunosignal;
wherein said CT immunosignal will decrease, relative to the CT immunosignal generated in the absence of Aβ42 oligomers, when Aβ42 oligomers are detected.

4. An assay of claim 3 wherein said bead based proximity assay is an AlphaLISA assay.

5. An assay of claim 3 wherein the donor beads are conjugated to streptavidin and the acceptor beads are conjugated to an anti-Aβ42 CT antibody.

6. An assay of claim 5 wherein the Aβ42 CT antibody is 12F4.

7. An assay of claim 3 further comprising analyzing the reaction mixture of part (b) in the presence of at least one test compound, wherein a compound that results in a CT immunosignal that is increased more than three standard deviations from the CT immunosignal of a control is an Aβ42 oligomer inhibitor.
